(19) United States
(12) Patent Application Publication
(10) Pub. No.: US 2010/0010300 A1
(43) Pub. Date: Jan. 14, 2010

(54) Device, System and Method for Orienting a Sensor In-Vivo

(75) Inventor: Zvika Gilad, Haifa (IL)

Correspondence Address: Pearl Cohen Zedek Latzer, LLP, 1500 Broadway, 12th Floor, New York, NY, 10036, US

(73) Assignee: GIVEN IMAGING LTD.

(21) Appl. No.: 11/794,571

(22) PCT Filed: Dec. 28, 2005
(86) PCT No.: PCT/IL05/01396

§ 371 Date: Jul. 2, 2007

Related U.S. Application Data
(60) Provisional application No. 60/640,095, filed on Dec. 30, 2004.

(51) Int. Cl. A61B 1/04

(52) U.S. Cl. 600/109

(57) ABSTRACT

A device, system, and method may orient a device in-vivo. A ballast or other weight may be rotatable or otherwise movable within a ballast chamber within a device. An induced magnetic field may be used to shift and/or rotate the ballast weight. Rotation and/or shifting of the ballast weight may position the in-vivo device in a specific orientation. The rotation and/or shifting of the ballast may be controlled by circuitry within the in-vivo device and/or by external signals transmitted to the in-vivo device.

### FIELD OF THE INVENTION

The present invention relates to in-vivo sensing, and more particularly orientation of an in-vivo sensor using ballast or other weight in an in-vivo device.

### BACKGROUND OF THE INVENTION

Sensing devices such as for example a swallowable imaging capsule or other suitable devices may be inserted (e.g., by swallowing) into for example the gastrointestinal (GI) tract or other body lumen and for example attached to a given position, or moved passively through the GI tract by peristalsis, while collecting sensory data of in-vivo areas. However, passive movement of objects through larger body lumens, such as for example, the stomach or the large intestine may be slow and unpredictable. Furthermore, the device may become trapped in a fold of the walls of the body lumen or in another location where movement of the device may be limited. In such a position, a sensing or imaging device may not have a sufficiently wide field of image and/or field of illumination to obtain images suitable for diagnostic purposes. In these cases monitoring and diagnosing larger body lumens such as for example the stomach or the large intestine may be not efficient because the direction of the imager or sensor and the orientation of the images captured may be limited by the orientation of the imager or the device as it rests in for example a large body lumen.

### SUMMARY OF THE INVENTION

Embodiments of the present invention provide a device, system and method for orienting a sensing device, for example an in-vivo imaging device, in-vivo. According to embodiments of the present invention ballast or another unit incorporated within the in-vivo device may react to forces, for example electromagnetic forces, that may cause the ballast to shift or rotate. In some embodiments of the present invention, current passing through conductive coils provide an electromagnetic force that may initiate shifting or rotation ballast for example within a ballast chamber. Shifting of ballast, may for example shift the orientation of the in-vivo sensing device so as to, for example change the field of view of an in-vivo imaging device. In other embodiments of the present invention, rotation of the ballast in-vivo may, for example function as a gyroscope and stabilize the orientation of the in-vivo device.

### BRIEF DESCRIPTION OF THE DRAWINGS

The present invention will be understood and appreciated from the following detailed description taken in conjunction with the drawings in which:

FIG. 1 is a simplified conceptual illustration of an in-vivo sensing system with a device that includes a ballast weight and a magnet, in accordance with an embodiment of the invention;

FIG. 2 is a schematic drawing of a ballast weight in a ballast chamber in accordance with an embodiment of the invention;

FIG. 3 is a depiction of the angles of orientation of a device being tilted while resting within a large body lumen in accordance with an embodiment of the invention;

FIG. 4 is a schematic drawing of an asymmetrical ballast weight in accordance with an embodiment of the present invention;

FIG. 5 is a schematic diagram of a spherical ballast weight in a tubular ballast chamber in accordance with an embodiment of the invention;

FIG. 6 is a flow chart of a method of changing the orientation of an imaging device in accordance with an embodiment of the invention; and

FIG. 7 is a schematic diagram of a device with a ballast weight askew of the vertical and horizontal planes of the device, in accordance with an embodiment of the invention;

It will be appreciated that for simplicity and clarity of illustration, elements shown in the figures have not necessarily been drawn accurately or to scale. For example, the dimensions of some of the elements may be exaggerated relative to other elements for clarity, or several physical components may be included in one functional block or element. Further, where considered appropriate, reference numerals may be repeated among the figures to indicate corresponding or analogous elements.

### DETAILED DESCRIPTION OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well-known features may be omitted or simplified in order not to obscure the present invention.

Some embodiments of the present invention are directed to a typically swallowable in-vivo device, e.g., a typically swallowable in-vivo sensing or imaging device. Devices according to embodiments of the present invention may be similar to embodiments described in U.S. patent application Ser. No. 09/800,470, entitled “Device and System for In-vivo Imaging”, filed on 8 Mar., 2001, published on Nov. 1, 2001 as United States Patent Application Publication Number 2001/0035902, and/or in U.S. Pat. No. 5,604,531 to Iddan et al., entitled “In-Vivo Video Camera System”, and/or in U.S. Patent application Ser. No. 10/046,541, filed on Jan. 16, 2002, published on Aug. 15, 2002 as United States Patent Application Publication Number 2002/0109774, all of which are hereby incorporated by reference. An external receiver/recorder unit, a processor and a monitor, e.g., in a workstation, such as those described in the above publications, may be suitable for use with some embodiments of the present invention. Devices and systems as described herein may have other configurations and/or other sets of components. For example, the present invention may be practiced using an endoscope, needle, stent, catheter, etc. Some in-vivo devices may be capsule shaped, or may have other shapes, for example, a peanut shape or tubular, spherical, conical, or other suitable shapes.

Some embodiments of the present invention may include, for example, a typically swallowable in-vivo device. In other embodiments, an in-vivo device need not be swallowable and/or autonomous, and may have other shapes or configurations. Some embodiments may be used in various body lumens, for example, the GI tract, blood vessels, the urinary tract, the reproductive tract, or the like. In some embodiments, the in-vivo device may optionally include a sensor, an imager, and/or other suitable components.

Embodiments of the in-vivo device are typically autonomous and are typically self-contained. For example, the in-vivo device may be or may include a capsule or other unit where all the components are substantially contained within a container, housing or shell, and where the in-vivo device does not require any wires or cables to, for example, receive power or transmit information. The in-vivo device may communicate with an external receiving and display system to provide display of data, control, or other functions. For example, power may be provided by an internal battery or a wireless receiving system. Other embodiments may have other configurations and capabilities. For example, components may be distributed over multiple sites or units. Control information may be received from an external source.

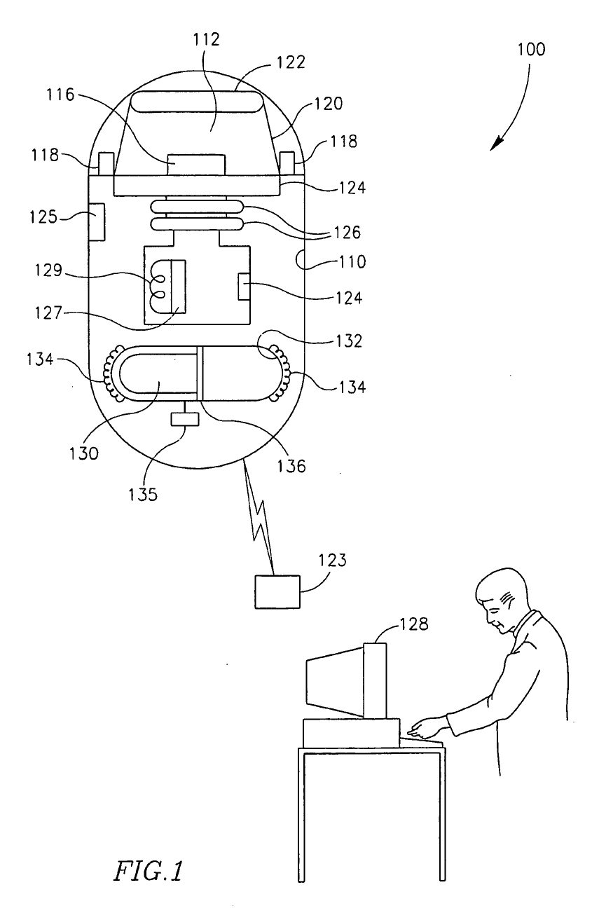

Reference is made to FIG. 1, which is a simplified conceptual illustration of an in-vivo sensing device including one or more ballasts, weights or other devices and a magnet such as for example, an electromagnet, in accordance with an embodiment of the invention. Device 100 may include a sensing unit 112, such as for example an imaging unit within an outer covering or housing 110, constructed and operative in accordance with an embodiment of the invention. Housing 110 may be, for example, spherical, ovoid, or any other suitable shape and may be partially deformable. Sensing unit 112 may include at least one sensor such as for example an image sensor 116, an optical system or lens 122 and a lens holder 120, which may be situated along an external wall of device 100, as well as one or more (e.g., a pair, a ring, etc.) of illumination sources 118, such as light emitting diodes (LEDs), which may illuminate the areas to be imaged by the image sensor 116. Image sensor 116 may be or may include a charge coupled device (CCD) or a complementary metal oxide semiconductor (CMOS) image sensor. Other suitable image sensors may be used. Other positions for image sensor 116 and more than one image sensor may be used and other shapes of a housing 110 may be used. Device 100 may include one or more circuit boards 124, and circuitry, for example that may include one or more switches, that may have a capacity to control or regulate one or more components in device 100, and one or more power sources 126 such as for example batteries. In some embodiments device 100 may include a transmitter 127, such as for example a wireless or radio transmitter, and an antenna 129. Device 100 may include a receiver 121 that may receive signals from an external source. Device 100 may transmit sensory data in the form of signals to an external receiver 123 where such signals or images may be stored or further processed for viewing on an external display 138 such as for example a monitor. In some embodiments, the transmitter 127 may for example transmit image signals to the external receiver 123 so that images may be viewed for example on-line or in real time. Display 138 or other display may in some embodiments of the present invention be integral to receiver 123. Other suitable viewing methods may be used. In other embodiments of the present invention an external transmitter 128 may transmit signals, for example, commands to device 100, for example to controller 137.

In some embodiments, device 100 may include one or more ballast(s) 130 that may be for example at least partially housed in a ballast chamber 136. Ballast chamber 136 need not be used. Ballast 130 may be fixed or moveable, for example rotatably fixed, held, and/or suspended on for example a pivot 136 in a ballast chamber 132. Other connection methods and positions for ballast(s) may be used. Ballast 130 may be, for example, a section of a cylinder, disk or sphere, such as a quarter or half cylinder. Shaping a section of ballast 130 as spherical or cylindrical may aid efficient movement within a container, but other suitable shapes, including non-rounded shapes, are possible. In some embodiments, ballast 130 may be flat, disk, or ring shaped, may be spherical or have other suitable shapes. Ballast 130 may have functionality other than a ballast; for example it may be a functional part of device 100. In one embodiment of the present invention, ballast 130 or ballast chamber 132 may be rotatable and may include one or more ballast units or weights. Some embodiments of the invention may be configured without a ballast chamber 132 and a pivot 136. One or more coils 134 together with, for example, a controller 137 or switch 135 may be used to control the position of ballast 130. Switch 135 may be for example a reed or MEMS switch or other suitable switch. Controller 137 may be any suitable controller, e.g. a controller that may be incorporated within a brushless motor. Device 100 may include a second or additional sensor 125 such as for example e.g. a blood detection sensor, pH sensor, electrical impedance sensor, pressure sensor, and temperature sensor, etc. Device 100 may be a swallowable autonomous device, and may be capsule shaped, but may have other forms, such as spherical.

Device 100 may be inserted into a body lumen for in-vivo imaging and it may be fixed at a position in the body or it may move through for example a GI tract or other body lumen.

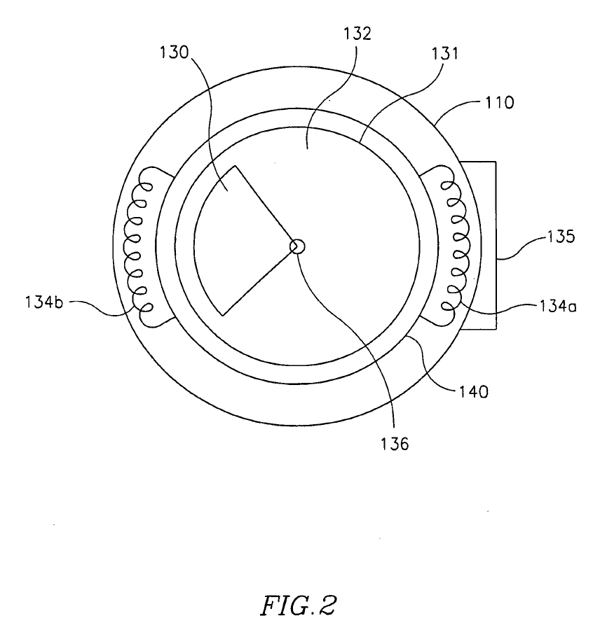

Reference is made to FIG. 2, a schematic drawing of a ballast weight in a ballast chamber in accordance with an embodiment of the invention. In some embodiments, ballast 130 may be configured in the shape of a ball, an arc, wedge or portion of a disk that may rotate and/or be made to rotate or shift in various positions within for example the ballast chamber 132. In some embodiments, ballast 130 may be attached at a center point or other suitable point to an axle or pivot 136 that may rotate within ballast chamber 132 such that ballast 130 may trace for example a circular orbit around the center of ballast chamber 132 as ballast 130 may be held by pivot 136. Ballast 130 or other weight may have a specific gravity of greater than the specific gravity of the entirety of device 100, and may cause at least an end or side of device 100 to sink or partially submerge in a liquid that may be found in a body lumen.

In some embodiments, ballast 130 may be or include a magnet or a metallic element that may be responsive to one or more magnetic forces such as for example an electromagnetic force. In some embodiments, ballast 130 may be a permanent magnet with a north and a south side. In some embodiments, one or more magnets, electromagnetic coils 134 or other components capable of generating an electromagnetic field from within device 100 may be configured on one or more sides or around the circumference of ballast chamber 132. In embodiments of the invention a plurality of magnets or coils 134 that may take on magnetic properties when current flows through them, may be activated, magnetized or demagnetized independently of one another. For example, coil 134a may at certain periods carry a positive current while coil 134b may carry a negative current. Currents may then be reversed between the two coils 134. Other numbers of coils 134 may be used and other alternating schedules for current in such coils 134 may be used. In some configurations, coils 134 may behave or function as the coils of an electrical motor, and ballast 130 may behave or function as a rotor. In some embodiments, a switch 135, such as for example a reed or MEMS switch or other suitable switch may be configured into for example a side of ballast chamber 132, and may connect the power source 126 to the conductive coils 134 and for example, control the flow of current into coils 134. In embodiments of the present invention, ballast 130 may be or include an electromagnet that may for example, carry currents in different directions, and coils 134 may be substituted with permanent magnets.

In some embodiments, one or more switches 135 may be used and/or a controller 137 may be used to control the current flow through coils 134. In other embodiments of the present invention controller 137 or at least part of its functionality may be external to device 100 and may transmit commands to device 100. Controller 137 may control the current flow through coils 134 and thus control the electromagnetic force and/or the direction of the electromagnetic force provided by the coils. Other switches may be used and other configurations for a rotor may be used. In some embodiments when a current may be passed through coils 134a and 134b, and for example alternated between positive and negative among such coils 134, ballast 130 may be propelled from, for example, one side of chamber 132 to another, or may rotate within ballast chamber 132. In other embodiments of the present invention, rotation or shifting of ballast 130 may be by a motor, other actuator, or by other suitable means. For example axle 136 may be fixed to a motor shaft or other rotating means. Other suitable rotating means may be implemented.

In some embodiments, ballast 130 may be or include one or more power sources 126, for example or other components of device 100 that may have one or more other roles or functions within device 100.

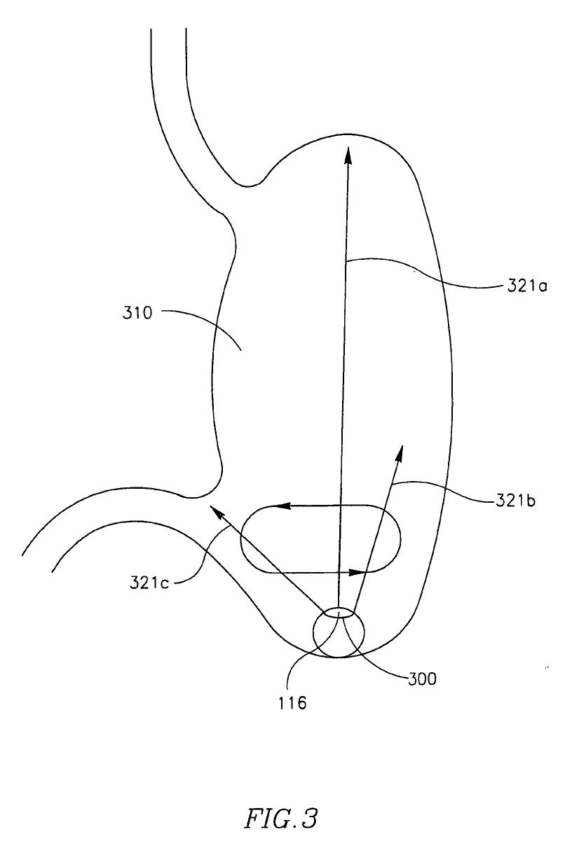

Reference is made to FIG. 3, a depiction of a device resting within a large body lumen in accordance with an embodiment of the invention. In operation, device 300 may come to rest, for example, in a stomach 310. In an initial state, image sensor 116 of device 300 may be oriented for example upwards 321a towards an upper portion of the stomach 310 such that images may be captured of such upper portion of stomach 310. At certain times or in response to a signal from for example an external source or a timer and/or controller 137 within device 300, ballast 130 may be tilted or moved within ballast chamber 132. The weight of ballast 130 may pivot or tilt device 100 to alter or change an orientation of image sensor 116 towards for example 321b, and image sensor 116 may capture images of another portion of the stomach 310 or other body lumen. As ballast 130 may be further rotated within ballast chamber 132, device 100 may tilt again towards another side or area of a body lumen such as for example 321c, and image sensor 116 may capture images of a further portion of a body lumen such as for example stomach 310. In some embodiments, as ballast 130 may alter the tilt of device 300, the field of view and orientation of image sensor 116 may trace an orbit around for example a center of gravity or a lowest point of device 300, as such orbit may be indicated by arrow 321d. Image sensor 116 may image a body lumen before, during, and after a change in the orientation of image sensor 116 or of device 100.

In some embodiments, the magnet, weight or ballast 130 may change the orientation of a sensing unit 112 by tilting the entire device 100. In some embodiments, ballast 130 and ballast chamber 132 may be attached to sensing unit 112, and sensing unit 112 may tilt with the movements of ballast 130, independent of the movements of the rest of housing 110, such as are described in embodiments of the invention in publication WO 2004/028336 filed on Sep. 30, 2003 and entitled “In Vivo Imaging System”, assigned to the common owner of this application and incorporated by reference herein.

Returning to FIG. 2, in some embodiments, ballast 130 may be a weighted object containing or including metallic elements such as iron, brass or other magnetically responsive materials. Other materials or mixtures of materials may be used.

In some embodiments, ballast 130 may be constructed in one piece with pivot 136 so that a rotation of ballast 130 may also rotate pivot 136. The ends of pivot 136 may be, for example, sharpened and fastened into holes, indentations or dimples at the top and bottom of chamber 132 which may allow pivot 136 to rotate. In some embodiments, pivot 136 may be fixed and a sleeve of ballast 130 may wrap around pivot 136 so that pivot 136 may remain stationary as ballast 130 rotates. In some embodiments, ballast may move freely within chamber 132.

In some embodiments, the inner or outer circumferential surface of chamber 132 may include or be fashioned of, for example, a flexible circuit board 140 onto which coils 134 may be attached and through which coils 134 may be connected to a power source 126 such as for example, via one or more switches 135 or controller 137. Flexible circuit board 140 may be configured as may be described in embodiments of the invention described in publication WO 02/102224 entitled “In-vivo Device with a Circuit Board having Rigid Sections and Flexible Sections,” and in U.S. patent application Ser. No. 10/879,054 filed on Jun. 30, 2004 and entitled “In-vivo Device having Flexible Circuit Board and Method of Manufacturing Thereof,” each assigned to the common assignee of this application and each incorporated by reference herein. In some embodiments, the circuit board 140 may be joined or folded into a closed loop onto which one or a plurality of coils 134 may be attached. Coils 134 may in some embodiments be attached to the inside or to the outside of, for example, circumferential walls 131 of chamber 132. Walls of chamber 132 may be or include other materials and may be constructed of components other than circuit board 140. In some embodiments the outside walls of chamber 132 may be at least partially inside walls of housing 110, and magnets or coils may be located elsewhere in device 100.

Ballast chamber 132 may in some embodiments occupy a circumference slightly smaller than the inside circumference of housing 110. Other sizes are possible, and in some embodiments chamber 132 may be significantly smaller than housing 110. Preferably, the height and circumference of chamber 132 may be slightly larger than the height and radial length or diameter of ballast 130 to permit ballast 130 to rotate fairly freely and evenly within chamber 132. Other shapes and configurations for a ballast or weight and a surrounding chamber, if used, are possible, and the terms “height” and “diameter” need not be applicable.

In some embodiments, the rate of rotation of ballast 130 may be slow, and a fairly few number of rotations of ballast 130, such as three or four per minute or in a particular body lumen, may be necessary to orient sensing unit 112 towards the various directions of such a body lumen necessary to capture images of such body lumen. In some embodiments the rate of rotation of ballast 130 may correspond to the frame rate of sensing unit 112 so as to let sensing unit 112 capture a sufficient number of images in each orientation of the imaging device 116. In other embodiments the rotation of ballast 130 may be implemented for other suitable purposes, for example, the rotating ballast may act as a gyroscope to maintain an orientation of device 100. In such an example, the rotation of ballast 130 may be at a relatively faster speed. According to one embodiment, the axis of rotation and other parameters, for example, the speed and/or direction of rotation may be controllable in-vivo or externally by external commands, for example commands transmitted by the external transmitter 128 to device 100. In some embodiments, a timer in device 100 may activate the rotation of ballast 130 in accordance with the estimated time that device 100 may be in a particular large volume lumen. In some embodiments a rotation of ballast 130 may not be a complete orbit of a given focal point, but may be a movement of the ballast 130 such as for example a partial rotation or a movement from side to side or from a side to a middle of for example a chamber 132.

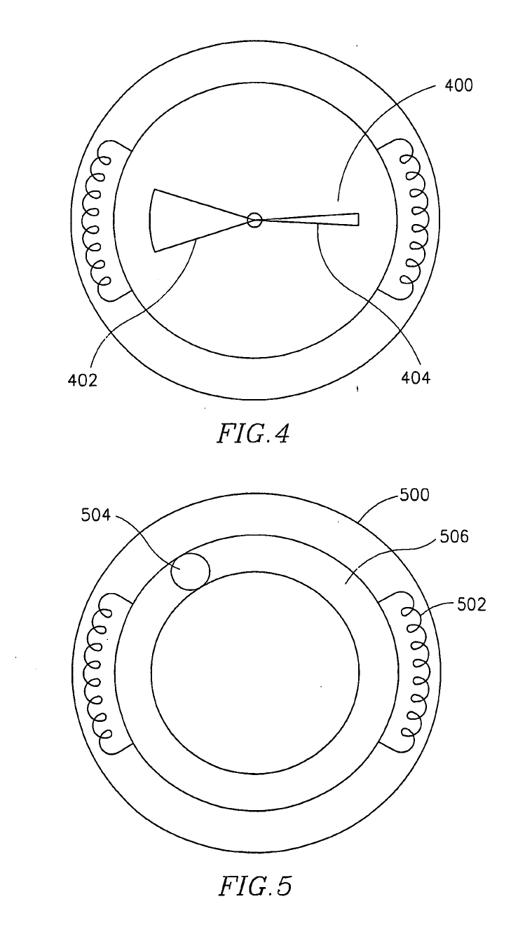

Reference is made to FIG. 4, a schematic drawing of an asymmetrical ballast in accordance with an embodiment of the present invention. In some embodiments, ballast 400 may be configured in a rotor-like shape but may be, for example, weighted unevenly between a “north” and a “south” side. For example, in some embodiments, a north side 402 of ballast 400 may be heavier than a south side 404 so that upon the rotation of rotor 400, device 100 or sensing unit 112 may tilt or wobble with the rotation of ballast 400.

In some embodiments, coils 134 may receive current, or be powered by an external energy source, in accordance with an embodiment of the present invention. A power supply of device 100 may include for example a conductive coil, configured for receiving energy from an external energy source, a rectifier circuit for converting AC voltage to DC voltage and a capacitor. A capacitor ranging from several mili-Farads to a few hundred mili-Farads may be used (other suitable ranges may be used) or alternatively, a chargeable battery (not shown) may be used for storage of the voltage required for operation of the electrical components of device 100. For example, a capacitor of about 10 Farad and 5 mWatt may be suitable for use in one embodiment of the present invention. In other embodiments of the present invention, coils 134 may receive current from an in-vivo power source, for example, from power source 126 (FIG. 1).

The device 100 may include components and operate similarly to the imaging systems described in U.S. Pat. No. 5,604,531 to Iddan, et al., WO 01/65995 and/or WO 02/054932, each assigned to the common assignee of the present application and each hereby incorporated by reference. Furthermore, a reception, processing and review system may be used, such as in accordance with embodiments of U.S. Pat. No. 5,604,531 to Iddan, et al., WO 01/65995 and/or WO 02/054932, although other suitable reception, processing and review systems may be used.

In some embodiments, ballast 130 may be attracted to or repelled from a current when a current is applied. A movement of ballast 130 either towards or away from a current may tilt or wobble a device 100.

Reference is made to FIG. 5, a schematic diagram of a spherical ballast in a tubular ballast chamber in accordance with an embodiment of the invention. In some embodiments, spherical ballast 504 may be made to roll through a circular or tubular ballast chamber 506 by one or more coils 502 within a ballast chamber 500. Ballast 504 may be or include a magnetic ball or other weighted sphere that may include elements reactive to magnetic forces. As the current in coils 502 may for example alternate between positive and negative, ballast 504 may be moved within chamber 506. In some embodiments, a ballast chamber 506 may be configured for example as a straight, curved or oblong tube. As ballast 130 may be drawn to or repulsed from a current, device 100 may move, tilt or pivot Chambers and ballast of other shapes and configurations may be used.

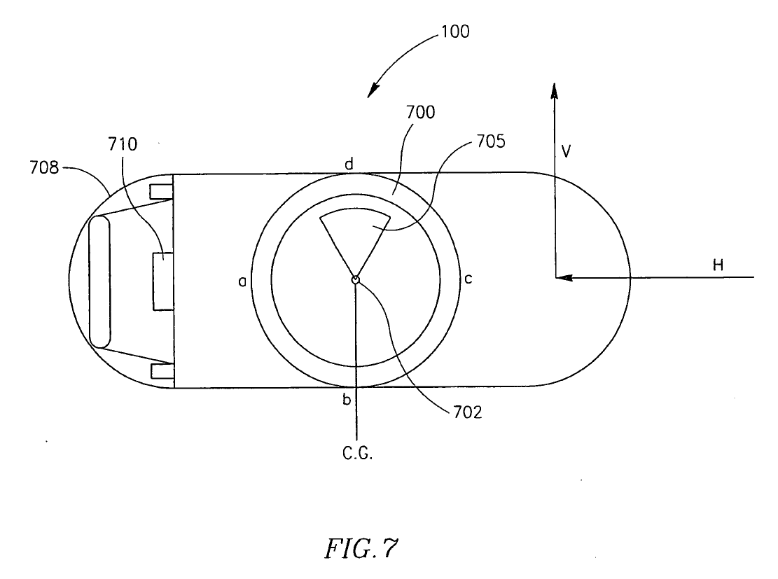

Reference is made to FIG. 7, a schematic diagram of a device with ballast askew of the vertical and horizontal planes of the device, in accordance with an embodiment of the invention. When used herein, vertical (V) and horizontal (H), and left and right, up and down, etc. are relative terms, and may be interchanged depending on the vantage point of the viewer and the orientation of the device, other terms may be used. Ballast chamber 700 may be configured so that its center point may be at the center of gravity of the pitch and yaw moments of the device 100. The placement of ballast chamber 700 may be angled so that it may be askew of both the V and H planes of device 100. As ballast 705 rotates within chamber 700 to on for example pivot 702, for example point ‘a’, ballast 705 may be anterior to the center of gravity (C.G.) of the pitch axis and to the right of the center of the yaw axis. As a result, a front end 708 of device 100 may pitch down and yaw to the right. As ballast 705 may continue to rotate towards point ‘b’, device 100 may pitch down and yaw to the center. As ballast 705 rotates toward point ‘c’, device 100 may pitch up and yaw to the left. As ballast continues toward point ‘d’, device may pitch down and yaw to the center. This pattern of movements may alter the orientation of an image sensor 710 as device 100 moves through a body lumen so that the field of view of the image sensor 710 may sweep up and down, and to the right and left. Such a pattern may permit an image sensor 710 in a fixed position within a device to capture images of the upper and lower, and/or lateral walls of a body lumen as the device 100 moves through the lumen. Other patterns of movement are possible and other configurations of movement of ballast 705 may be possible.

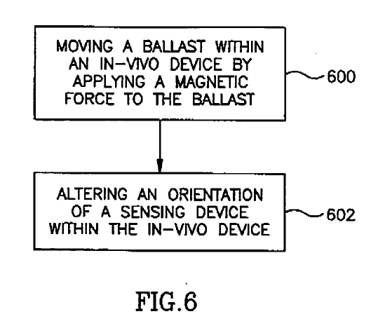

Reference is made to FIG. 6, a flow chart of a method of changing the orientation of an imaging device in accordance with an embodiment of the invention. In block 600, a ballast or other device within an in-vivo device, for example, a metallic or magnetically reactive ballast 130, 400, 504, and/or 705 within an in-vivo device 110 may be moved by a force, for example an electromagnetic force applied, for example, from within such in-vivo device. The magnetic force may be generated by one or more magnets such as for example electrical coils 134 and/or 502 that may be supplied with current and that may be placed around or adjacent to a chamber 132, 506 and/or 700 in which such ballast may be moveably held. In some embodiments the current in one or more of such coils 134 and/or 502 may alternate to attract or repel such ballast. In some embodiments the ballast 130, 400, 504, and/or 705 may rotate within the ballast chamber 132, 506, and/or 700. In block 602, an orientation of a sensor and/or the in vivo device 100, for example, image sensor 116 and/or 710 in the in-vivo device 100 in which such ballast 130, 400, 504, and/or 705 may be encapsulated may be altered as the weight of the ballast 130, 400, 504, and/or 705 changes the tilt or position of the in-vivo device 110. In some embodiments, the ballast 130, 400, 504, and/or 705 may be attached to the sensor, for example, image sensor 116 and/or 710 so that the orientation of the sensor may be changed without tilting the body of the in-vivo device 100. In other embodiments of the present invention, the ballast may be rotated in a specified orientation such that the orientation of the in-vivo device 100 may be stabilized. In some embodiments of the present invention the axis of rotation of the ballast may be altered or controlled, for example by controller 137. In other embodiments of the present invention the speed of rotation of the ballast may be controlled, for example by controller 137. Other suitable operations or series of operations may be used.

A device, system and method in accordance with some embodiments of the invention may be used, for example, in conjunction with a device which may be inserted into a human body. However, the scope of the present invention is not limited in this regard. For example, some embodiments of the invention may be used in conjunction with a device which may be inserted into a non-human body or an animal body.

While the present invention has been described with reference to one or more specific embodiments, the description is intended to be illustrative as a whole and is not to be construed as limiting the invention to the embodiments shown. It is appreciated that various modifications may occur to those skilled in the art that, while not specifically shown herein, are nevertheless within the true spirit and scope of the invention.

I claim:

1. An in-vivo sensing device comprising:
a sensing unit;
a moveable ballast; and
a conductive coil.

2. The in-vivo sensing device according to claim 1 wherein the sensing unit comprises:
an image sensor; and
an illumination source.

3. The in-vivo sensing device according to claim 1 comprising a ballast chamber wherein the ballast is at least partially enclosed within the ballast chamber.

4. The in-vivo sensing device according to claim 3 wherein the conductive coil is positioned on a surface defined by the ballast chamber.

5. The in-vivo sensing device according to claim 1 comprising a pivot wherein the ballast is suspended on the pivot.

6. The in-vivo sensing device according to claim 1 wherein the ballast is rotatable.

7. The in-vivo sensing device according to claim 1 wherein the ballast comprises a magnet.

8. The in-vivo sensing device according to claim 1 comprising a switch wherein the switch is to connect the power source to the conductive coil.

9. The in-vivo sensing device according to claim 1 wherein the ballast includes at least a rotor.

10. An in-vivo sensing system comprising:
an in-vivo device comprising a sensing unit and a ballast wherein the ballast is movable within the in-vivo device; and
an external receiver.

11. The in-vivo sensing system according to claim 10 comprising a controller.

12. The in-vivo sensing system according to claim 11 comprising an external transmitter wherein the transmitter is to transmit commands to the controller.

13. The in-vivo sensing system according to claim 10 wherein the ballast comprises a magnet.

14. The in-vivo sensing system according to claim 10 wherein the ballast comprises a rotor.

15. The in-vivo sensing system according to claim 11 comprising an electromagnet.

16. The in-vivo sensing system according to claim 15 wherein the electromagnet is housed within the in-vivo device and the electromagnet is at least partially controlled externally from the in-vivo device.

17. A method for in-vivo sensing comprising:
exerting a force on a ballast wherein the ballast is within an in-vivo device; and
moving the ballast within the in-vivo device.

18. The method according to claim 17 wherein the force is an electromagnetic force.

19. The method according to claim 17 wherein exerting a force is by supplying current to a conductive coil.

20. The method according to claim 17 wherein the in-vivo device is an imaging device.

21. The method according to claim 17 comprising changing the orientation of the in-vivo device.

22. The method according to claim 17 comprising stabilizing the orientation of the in-vivo device.

23. The method according to claim 17 comprising controlling the axis of rotation of the ballast.

24. The method according to claim 17 comprising controlling the speed of ration of the ballast.
